(19) United States
(12) Patent Application Publication
(10) Pub. No.: US 2014/0309256 A1
(43) Pub. Date: Oct. 16, 2014

(54) Therapeutic Method

(71) Applicant: FibroGen, Inc., San Francisco, CA (US)

(72) Inventors: Kin-Hung Peony Yu, Hillsborough, CA (US); Thomas B. Neff, Atherton, CA (US)

(21) Appl. No.: 14/357,131

(22) PCT Filed: Nov. 8, 2012
(86) PCT No.: PCT/US12/64140

§ 371 Date: May 8, 2014

Related U.S. Application Data
(60) Provisional application No. 61/557,784, filed on Nov. 9, 2011.

(51) Int. Cl. A61K 31/4725

(52) U.S. Cl. CPC A61K 31/4725
USPC 514/309

(57) ABSTRACT

The present invention provides a method of treatment for anemia, or of increasing the hemoglobin level, in a subject without significantly increasing the platelet count during the course of treatment, by administering a compound that inhibits HIF prolyl hydroxylase.

### FIELD OF THE INVENTION

The present invention relates to methods useful for treating anemia or increasing hemoglobin without increasing platelet count.

### BACKGROUND OF THE INVENTION

For more than twenty years, recombinant human erythropoietin (rhEPO) and other erythropoiesis-stimulating agents (ESAs) have been widely used for treatment of anemia in patients with chronic kidney disease (CKD) and in cancer patients receiving chemotherapy. Several recent studies have reported increased mortality and cardiovascular events when ESAs were administered to CKD patients (Kowalczyk et al. Med. Sci. Monit. 2011 17:RA240; Fishbane and Besarab Clin, J. Am. Soc. Nephrol. 2007 2:1274) ESAs have long been known to increase platelet number (Dahl et al. Semin. Dialysis 2008 21:210; Kaupke et al. J. Am. Soc. Nephrol. 1993 3:1672; Stohlawetz et al. Blood 2000 95:2983; Homoncik et al. Aliment. Pharmacol. Ther. 2004 20:437) and frequently lead to functional or absolute iron deficiency. Increased platelet number, whether from iron deficiency or from other causes, may increase the risk of thrombovascular events and lead to increased mortality (Khorana et al. Cancer 2005 104:2822; Streja et al. Am. J. Kidney Dis. 2008 52:727). There is thus a need for methods for treating anemia which do not carry an associated risk for increased platelet count and the resulting risk of thrombovascular events.

### SUMMARY OF THE INVENTION

The invention relates to a method of treating anemia in a subject in need thereof without significantly increasing the platelet count in the subject, the method comprising administering to the subject a therapeutically effective amount of a compound that inhibits HIF prolyl hydroxylase. In another embodiment the invention relates to a method of increasing hemoglobin in a subject in need thereof without significantly increasing the platelet count in the subject, the method comprising administering to the subject a therapeutically effective amount of a compound that inhibits HIF prolyl hydroxylase.

In a separate embodiment, the invention relates to a method of maintaining the platelet count in a subject in need of treatment for anemia, the method comprising administering to the subject a therapeutically effective amount of a compound that inhibits HIF prolyl hydroxylase. In a further embodiment, the invention relates to a method of maintaining the platelet count in a subject in need of an increase in hemoglobin, the method comprising administering to the subject a therapeutically effective amount of a compound that inhibits HIF prolyl hydroxylase.

In another embodiment, the invention relates to a method of decreasing the platelet count in a subject in need of treatment foranemia, the method comprising administering to the subject a therapeutically effective amount of a compound that inhibits HIF prolyl hydroxylase. In yet another embodiment, the invention relates to a method of decreasing the platelet count in a subject in need of an increase in hemoglobin, the method comprising administering to the subject a therapeutically effective amount of a compound that inhibits HIF prolyl hydroxylase.

In a further embodiment, the invention relates to a method of increasing hemoglobin and decreasing the platelet count in a subject with low hemoglobin, the method comprising administering to the subject a therapeutically effective amount of a compound that inhibits HIF prolyl hydroxylase. In another embodiment, the invention relates to a method of treating anemia and decreasing the platelet count in a subject having anemia, the method comprising administering to the subject a therapeutically effective amount of a compound that inhibits HIF prolyl hydroxylase.

In these and other embodiments of the method of the invention described herein, the compound that inhibits HIF prolyl hydroxylase preferably is (4-hydroxy-1-methyl-7-phenoxy-isoquinoline-3-carbonyl)amino]-acetic acid. Other compounds that inhibit HIF prolyl hydroxylase are known and described herein.

The present invention also provides a compound that inhibits HIF prolyl hydroxylase for use in treating anemia without significantly increasing platelet count, for use in increasing hemoglobin without significantly increasing platelet count, for use in maintaining the platelet count in a subject treated for anemia, for use in maintaining the platelet count in a subject in need of an increase in hemoglobin, for use in decreasing the platelet count in a subject treated for anemia, for use in decreasing the platelet count in a subject in need of an increase in hemoglobin, for use in increasing hemoglobin and decreasing the platelet count in a subject with low hemoglobin, and for use in treating anemia and decreasing the platelet count in a subject having anemia.

The present invention also provides (4-hydroxy-1-methyl-7-phenoxy-isoquinoline-3-carbonyl)-amino]-acetic acid for use in treating anemia without significantly increasing platelet count, for use in increasing hemoglobin without significantly increasing platelet count, for use in maintaining the platelet count in a subject treated for anemia, for use in maintaining the platelet count in a subject in need of an increase in hemoglobin, for use in decreasing the platelet count in a subject treated for anemia, for use in decreasing the platelet count in a subject in need of an increase in hemoglobin, for use in increasing hemoglobin and decreasing the platelet count in a subject with low hemoglobin, and for use in treating anemia and decreasing the platelet count in a subject having anemia.

The present invention also provides a compound that inhibits HIF prolyl hydroxylase for use in the preparation of a medicament for treating anemia without significantly increasing platelet count, for increasing hemoglobin without significantly increasing platelet count, for maintaining the platelet count in a subject treated for anemia, for maintaining the platelet count in a subject in need of an increase in hemoglobin, for decreasing the platelet count in a subject treated for anemia, for decreasing the platelet count in a subject in need of an increase in hemoglobin, for increasing hemoglobin and decreasing the platelet count in a subject with low hemoglobin, and for treating anemia and decreasing the platelet count in a subject having anemia.

The present invention also provides (4-hydroxy-1-methyl-7-phenoxy-isoquinoline-3-carbonyl)-amino]-acetic acid for use in the preparation of a medicament for treating anemia without significantly increasing platelet count, for increasing hemoglobin without significantly increasing platelet count, for maintaining the platelet count in a subject treated for anemia, for maintaining the platelet count in a subject in need of an increase in hemoglobin, for decreasing the platelet count in a subject treated for anemia, for decreasing the platelet count in a subject in need of an increase in hemoglobin, for increasing hemoglobin and decreasing the platelet count in a subject with low hemoglobin, and for treating anemia and decreasing the platelet count in a subject having anemia.

These and other embodiments of the present invention will readily occur to those of skill in the art in light of the disclosure herein, and all such embodiments are specifically contemplated.

### BRIEF DESCRIPTION OF THE DRAWINGS

FIGS. 1A and 1B. Mean hemoglobin levels (g/dL) in subjects treated with Compound A in Groups A and B (FIG. 1A), or Groups C and D (FIG. 1B) over the course of treatment for 16 weeks (Groups A and B) or 24 weeks (Groups C and D). Last observation carried forward method was used to impute missing values.

FIG. 2. Mean platelet counts in subjects treated with Compound A in Groups A, B, C and D over the course of treatment for 16 weeks (Groups A and B) or 24 weeks (Groups C and D). Last observation carried forward method was used to impute missing values.

FIG. 3. Mean platelet counts in subjects treated with Compound A in all Groups A, B, C and D combined over the course of treatment for 16 weeks (Groups A and B) or 24 weeks (Groups C and D) stratified by quartile.

### DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items. It must be noted that as used herein and in the appended claims, the singular forms “a,” “an,” and “the” include plural references unless context clearly dictates otherwise.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed, Springer Verlag.

The section headings are used herein for organizational purposes only, and are not to be construed as in any way limiting the subject matter described herein.

The present inventors have surprisingly discovered that certain small molecule inhibitors of HIF prolyl hydroxylase, when administered for treatment of anemia and/or for increasing hemoglobin in subjects in need of such therapy, has the added beneficial effect of not significantly increasing the platelet count in the treated subjects. In particular, the small molecule inhibitor of HIF prolyl hydroxylase for use in the method of the invention is (4-hydroxy-1-methyl-7-phenoxy-isoquinoline-3-carbonyl)-amino]-acetic acid. The platelet count in subjects treated in the method of the invention is maintained or even decreased over the course of treatment. For subjects having a higher (high normal range) baseline platelet count, treatment with the compound (4-hydroxy-1-methyl-7-phenoxy-isoquinoline-3-carbonyl)-amino]-acetic acid typically decreases the platelet count over the course of treatment. For subjects having lower (low normal range) baseline platelet counts, treatment with the compound (4-hydroxy-1-methyl-7-phenoxy-isoquinoline-3-carbonyl)-amino]-acetic acid typically maintains or does not significantly increase the platelet count over the course of treatment. Particularly, for subjects having a baseline platelet count of greater than 300,000/ul, or greater than 320,000/ul, or greater than 340,000/ul, or greater than 360,000/ul, or greater than 380,000/ul, or between 300,000 and 400,000/ul, the compound (4-hydroxy-1-methyl-7-phenoxy-isoquinoline-3-carbonyl)-amino]-acetic acid can be used for decreasing the platelet count in subjects treated for anemia, or in subjects treated to increase hemoglobin. For subjects having a baseline platelet count of greater than 150,000/ul, or greater than 200,000/ul, or greater than 220,000/ul, or greater than 240,000/ul, or greater than 260,000/ul, or greater than 280,000/ul, or between 150,000 and 300,000/ul, the compound (4-hydroxy-1-methyl-7-phenoxy-isoquinoline-3-carbonyl)-amino]-acetic acid can be used for maintaining the platelet count in subjects treated for anemia, or in subjects treated to increase hemoglobin. This is in contrast to current methods for treating anemia and/or increasing hemoglobin by administering an erythropoiesis-stimulating agent (“ESA”), such as recombinant human erythropoietin (rHuEPO), e.g. epoetin α, epoetin beta, which have been shown to increase platelet count in treated subjects.

Accordingly, the present invention relates to methods of treating anemia or of increasing hemoglobin, in subjects in need of such therapy, without significantly increasing the platelet count in the treated subjects, by administering a therapeutically effective amount of a compound that inhibits HIF prolyl hydroxylase. In a particular embodiment, the invention relates to a method of treating anemia or of increasing hemoglobin, in subjects in need of such therapy, without significantly increasing the platelet count in the treated subjects, by administering a therapeutically effective amount of (4-hydroxy-1-methyl-7-phenoxy-isoquinoline-3-carbonyl)-amino]-acetic acid.

The present invention also provides a method of maintaining the platelet count in a subject in need of treatment for anemia by administering a therapeutically effective amount of a compound that inhibits HIF prolyl hydroxylase. In a particular embodiment, the invention provides a method of maintaining the platelet count in a subject in need of treatment for anemia by administering a therapeutically effective amount of (4-hydroxy-1-methyl-7-phenoxy-isoquinoline-3-carbonyl)-amino]-acetic acid. The present invention also provides a method of decreasing the platelet count in a subject in need of treatment for anemia by administering a therapeutically effective amount of a compound that inhibits HIF prolyl hydroxylase. In a particular embodiment, the invention provides a method of decreasing the platelet count in a subject in need of treatment for anemia by administering a therapeutically effective amount of (4-hydroxy-1-methyl-7-phenoxy-isoquinoline-3-carbonyl)-amino]-acetic acid.

The present invention also provides a method of maintaining the platelet count in a subject in need of an increase in hemoglobin by administering a therapeutically effective amount of a compound that inhibits HIF prolyl hydroxylase. In a particular embodiment, the invention provides a method of maintaining the platelet count in a subject in need of an increase in hemoglobin by administering a therapeutically effective amount of (4-hydroxy-1-methyl-7-phenoxy-isoquinoline-3-carbonyl)-amino]-acetic acid. The present invention also provides a method of decreasing the platelet count in a subject in need of an increase in hemoglobin by administering a therapeutically effective amount of a compound that inhibits HIF prolyl hydroxylase. In a particular embodiment, the invention provides a method of decreasing the platelet count in a subject in need of an increase in hemoglobin by administering a therapeutically effective amount of (4-hydroxy-1-methyl-7-phenoxy-isoquinoline-3-carbonyl)-amino]-acetic acid.

The present invention further provides a method of increasing hemoglobin and decreasing the platelet count in a subject with low hemoglobin comprising administering to the subject a therapeutically effective amount of a compound that inhibits HIF prolyl hydroxylase. In a particular embodiment, the invention provides a method of increasing hemoglobin and decreasing the platelet count in a subject with low hemoglobin comprising administering to the subject a therapeutically effective amount of (4-hydroxy-1-methyl-7-phenoxy-isoquinoline-3-carbonyl)-amino]-acetic acid. The invention additionally provides a method of treating anemia and decreasing the platelet count in a subject having anemia comprising administering to the subject a therapeutically effective amount of a compound that inhibits HIF prolyl hydroxylase. In a particular embodiment, the invention provides a method of treating anemia and decreasing the platelet count in a subject having anemia comprising administering to the subject a therapeutically effective amount of (4-hydroxy-1-methyl-7-phenoxy-isoquinoline-3-carbonyl)-amino]-acetic acid.

### DEFINITIONS

The term “anemia” as used herein refers to any abnormality in hemoglobin or erythrocytes that leads to reduced oxygen levels in the blood. Anemia can be associated with abnormal production, processing, or performance of erythrocytes and/or hemoglobin. The term anemia refers to any reduction in the number of red blood cells and/or level of hemoglobin in blood relative to normal blood levels.

Anemia can arise due to conditions such as acute or chronic kidney disease, infections, inflammation, cancer, irradiation, toxins, diabetes, and surgery. Infections may be due to, e.g., virus, bacteria, and/or parasites, etc. Inflammation may be due to infection, autoimmune disorders, such as rheumatoid arthritis, etc. Anemia can also be associated with blood loss due to, e.g., stomach ulcer, duodenal ulcer, hemorrhoids, cancer of the stomach or large intestine, trauma, injury, surgical procedures, etc. Anemia is further associated with radiation therapy, chemotherapy, and kidney dialysis. Anemia is also associated with HIV-infected patients undergoing treatment with azidothymidine (zidovudine) or other reverse transcriptase inhibitors, and can develop in cancer patients undergoing chemotherapy, e.g., with cyclic cisplatin- or non-cisplatin-containing chemotherapeutics. Aplastic anemia and myelodysplastic syndromes are diseases associated with bone marrow failure that result in decreased production of erythrocytes. Further, anemia can result from defective or abnormal hemoglobin or erythrocytes, such as in disorders including microcytic anemia, hypochromic anemia, etc. Anemia can result from disorders in iron transport, processing, and utilization, see, e.g., sideroblastic anemia, etc.

A “therapeutically effective amount” or dose of a compound, agent, or drug of the present invention refers to an amount or dose of the compound, agent, or drug that results in amelioration of symptoms or a prolongation of survival in a subject. Toxicity and therapeutic efficacy of such molecules can be determined by standard pharmaceutical procedures in cell cultures or experimental animals, e.g., by determining the LD50 the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The therapeutically effective amount is the amount of the compound or pharmaceutical composition that will elicit the biological or medical response of a tissue, system, animal, or human that is being sought by the researcher, veterinarian, medical doctor, or other clinician, e.g., an increase in hemoglobin levels, an increase in hematocrit, treatment of anemia, an increase in quality of life, etc.

A “low hemoglobin level” refers to a level of hemoglobin in a subject that is below the normal range for the particular subject. Normal hemoglobin range varies with species, gender, age, and several other factors. For example, in humans, normal hemoglobin levels range from 13 g/dL-18 g/dL for males and 12 g/dL-16 g/dL for females. Competent medical practitioners are well qualified to determine the appropriate normal hemoglobin range for any individual subject and to determine the particular hemoglobin level of the subject by methods that are well known in the art, some of which are described herein. A low hemoglobin level for adult human subject may be a hemoglobin level that is less than 13 g/dL, or less than 12 g/dL, or less than 11.5 g/dL, or less than 11 g/dL, or less than 10.5 g/dL, or less than 10 g/dL, or less than 9.5 g/dL, or less than 9.0 g/dL, or less than 8.5 g/dL.

Normal platelet count for human subjects is typically between 150,000 and 400,000/ul. High normal platelet count is typically between 300,000 and 400,000/ul; low normal platelet count is typically between 150,000 and 300,000/ul.

The term “HIFα” refers to the alpha subunit of hypoxia inducible factor protein. HIFα may be any human or other mammalian protein, or fragment thereof, including human HIF-1α (Genbank Accession No Q16665), HIF-2α (Genbank Accession No AAB41495), and HIF-3α (Genbank Accession No. AAD22668); murine HIF-1α (Genbank Accession No. Q61221), HIF-2α (Genbank Accession No. BAA20130 and AAB41496), and HIF-3α (Genbank Accession No. AAC72734); rat HIF-1α (Genbank Accession No. CAA70701), HIF-2α (Genbank. Accession No. CAB96612), and HIF-3α (Genbank Accession No. CAB96611); and bovine HIF-1α (Genbank Accession No. BA A78675). HIFα may also be any non-mammalian protein or fragment thereof, including Xenopus laevis HIF-1α (Genbank Accession No. CAB96628), Drosophila melanogaster HIF-1α (Genbank Accession No. JC4851), and chicken HIF-1α (Genbank Accession No. BAA34234). HIFα gene sequences may also be obtained by routine cloning techniques, for example by using all or part of a HIFα gene sequence described above as a probe to recover and determine the sequence of a HIFα gene in another species.

The terms “HIF prolyl hydroxylase” and “HIF PH” refer to any enzyme capable of hydroxylating a proline residue in the HIF protein, particularly in the HIFα subunit. Preferably, the proline residue hydroxylated by HIF PH includes the proline found within the motif LXXLAP, e.g., as occurs in the human HIF-1α native sequence at L₃₉₇TLLAP and L₅₅₉EMLAP. HIF prolyl hydroxylases (HPHs), also referred to as prolyl hydroxylase domain (PHD) proteins, or EGLN proteins, form an evolutionarily conserved subfamily of dioxygenases that uses oxygen and 2-oxoglutarate (2-00) as co-substrates, and iron and ascorbate as cofactors (Fong and Takeda, Cell Death and Differentiation 2008 15:635). Mammals have four members belonging to this subfamily, including PHD1/EGLN2/HPH3, PHD2/EGLN1/HPH2, PHD3/EGLN3/HPH1, and a recently characterized protein named P4H-TM (Fong, supra; Koivunen et al. J. Biol. Chem. 2007 282:30544). HIF PH includes members of the Egl-Nine (EGLN) gene family described by Taylor (2001, Gene 275:125-132), and characterized by Aravind and Koonin (2001, Genome Biol 2:RESEARCH0007), Epstein et al. (2001, Cell 107:43-54), and Bruick and McKnight (2001, Science 294:1337-1340). Examples of HIF PH enzymes include human SM-20 (EGLN1) (GenBank Accession No. AAG33965; Dupuy et al. (2000) Genomics 69:348-54), EGLN2 isoform 1 (GenBank Accession No. CAC42510; Taylor, supra), EGLN2 isoform 2 (GenBank Accession No. NP_{—}060025), and EGLN3 (GenBank Accession No. CAC42511; Taylor, supra); mouse EGLN1 (GenBank Accession No. CAC42515), EGLN2 (GenBank Accession No. CAC42511), and EGLN3 (SM-20) (GenBank Accession No, CAC42517); and rat SM-20 (GenBank Accession No. AAA19321). Additionally, HIF PH may include Caenorhabditis elegans EGL-9 (GenBank Accession No. AAD56365) and Drosophila melanogaster CG1114 gene product (GenBank Accession No AAF52050). HIF PH also includes any fragment of the foregoing full-length proteins that retain at least one structural or functional characteristic.

A compound that inhibits HIF prolyl hydroxylase is one that effectively reduces, diminishes, or eliminates the ability of the HIF prolyl hydroxylase enzyme(s) to hydroxylate the HIFα subunit. Inhibition of HIF prolyl hydroxylase can result in stabilization of HIF and transactivation of HIF target genes, for example, erythropoietin. Compounds that inhibit HIF prolyl hydroxylase are well known in the art and are described in, inter alia, U.S. Pat. Nos. 5,658,933; 5,620,995; 5,719,164; 5,726,305; 6,093,730; U.S. application Ser. No. 12/544,861; U.S. Patent Application Publication Nos. 2006/0199836; 2007/0298104; 2008/0004309; and PCT publication Nos. WO2009/073669; WO2009/089547; WO2009/100250; U.S. Patent Application Publication 2003/0176317, U.S. Patent Application Publication 2003/0153503, U.S. Pat. No. 7,323,475, U.S. Patent Application Publication 2006/0199836, U.S. Pat. No. 7,928,120, U.S. Pat. No. 7,696,223, U.S. Patent Application Publication 2010/0303928, U.S. Patent Application Publication 2010/0330199, U.S. Patent Application Publication 2010/0331400, U.S. Patent Application Publication 2010/0047367, PCT Application No. PCT/US2009/064065, U.S. Pat. No. 7,897,612. U.S. Pat. No. 7,608,621, U.S. Pat. No. 7,728,130, U.S. Pat. No. 7,635,715, U.S. Pat. No. 7,569,726, U.S. Pat. No. 7,811,595.

For use in some embodiments of the methods and medicaments of the present invention, the compound that inhibits HIF prolyl hydroxylase is preferably (4-hydroxy-1-methyl-7-phenoxy-isoquinoline-3-carbonyl)-amino]-acetic acid (Compound A).

### Method/Uses

The methods of the present invention of treating anemia or of increasing hemoglobin achieve the therapeutic goal (i.e., correct the anemia or increase the hemoglobin) without the deleterious side effect of increasing the platelet count, as is commonly seen in treatments using ESAs. Normal platelet count for healthy human subjects is between 150,000 and 400,000/μl, By “without significantly increasing the platelet count” is intended that the platelet count in the treated subjects does not significantly increase over the course of the treatment compared to the baseline pre-treatment platelet count. The “course of treatment” includes the time from the administration of the first dose of compound to the last dose of compound. For purposes of the present invention, a significant increase in platelet count is an increase of more than 10% from the baseline pre-treatment platelet count over the course of the treatment. The method of the present invention of treating anemia in a subject without significantly increasing the platelet count in the subject thus achieves the therapeutic goal of treating the anemia while not increasing the platelet count in the treated subject by more than 10% over the baseline pretreatment platelet count. The method of the present invention of increasing hemoglobin in a subject without significantly increasing the platelet count in the subject thus achieves the therapeutic goal of increasing the hemoglobin while not increasing the platelet count in the treated subject by more than 10% over the baseline pre-treatment platelet count.

In the methods of the present invention the platelet count of treated subjects remains substantially the same (i.e., an increase in platelet count of 10% or less) as the pre-treatment baseline platelet count, and in fact the platelet count may decrease from the baseline platelet count over the course of treatment. Measurement of the platelet count is typically done at intervals over the course of treatment, beginning with a baseline pre-treatment measurement. Measurements of the platelet count at intervals during the course of treatment are compared to the baseline pre-treatment platelet count to determine an increase or decrease. In the methods of the present invention, the platelet count measured at the end of the course of treatment is not significantly increased compared to the baseline pre-treatment platelet count. In some embodiments the platelet count measured at any interval during the course of treatment is not significantly increased compared to the baseline pre-treatment platelet count.

Techniques for measuring platelet counts are routine in the art and any conventional method for measuring platelet count may be used for the present methods. Different manual methods are available for determining platelet counts, including evaluation of blood smears and methods using erythrocyte-lysing agents followed by platelet counting in a counting chamber. Manual methods, however, are time-consuming and dependent of the skill of the operator. Furthermore, a rapid evaluation of platelet count is desirable for studies of platelet aggregation, in which fresh blood is essential for reliable results and platelet concentrations must be standardized. In addition, automated systems determine parameters such as mean platelet volume. Several automated cell counting systems using different techniques are available for determining platelet counts in whole blood. Impedance analyzers dilute blood cells in an electrically conductant medium and pass the cells through a small aperture between 2 electrodes. A change in electrical impedance that is proportional to the size of the cell is generated every time a cell passes the aperture. Platelet count measured with flow cytometers often is based on interruptions in a light (laser) beam, and change in light scatter give information about the size of the cell. Thus, platelets and red blood cells are separated on the basis of size in impedance analyzers as well as in some flow cytometers. In most analyzers, the different cell types are identified by using fixed settings of size. Some analyzers, however, are able to define the site limits on the basis of the distribution of cells in the given sample, Finally, flow cytometers are able to obtain precise platelet counts using specific platelet antibodies and color indicators.

The present invention also relates to methods of maintaining the platelet count in a subject in need of treatment for anemia or in need of an increase in hemoglobin. By “maintaining the platelet count” is intended that the platelet count in treated subjects does not increase by more than 10% from the baseline pre-treatment value over the course of treatment. In most cases, in the present method of maintaining the platelet count, the platelet count in treated subjects increases by 5% or less from the baseline pre-treatment value. In the present method of maintaining the platelet count, the platelet count in treated subjects may decrease but typically decreases by 5% or less from the baseline pre-treatment value. In the methods of maintaining the platelet count of the present invention, the suitable subjects are ones in need of treatment for anemia or in need of an increase in hemoglobin. Such subjects may be ones undergoing a treatment for anemia or undergoing a treatment for increasing hemoglobin. These and other suitable subjects are further described herein. The invention contemplates that the maintaining of the platelet count and the treatment for anemia and/or for increasing hemoglobin are simultaneously achieved by the administration of the compound of the invention.

The invention also relates to methods of decreasing the platelet count in subjects in need of treatment for anemia or in need of an increase in hemoglobin. By “decreasing the platelet count” is intended that the platelet count in the treated subjects decreases by more than 5% from the baseline pre-treatment value over the course of the treatment. The platelet count in subjects treated in the present method may decrease by 10%, or by 20%, or more from the baseline pre-treatment value over the course of the treatment. Typically, the higher the baseline platelet count, the larger the decrease will be. However, the decrease in platelet count that is achieved in the methods of the present invention is not such that the platelet count falls below the normal range, i.e., the platelet count does not fall below 150,000/μl. In the methods of decreasing the platelet count of the present invention, the suitable subjects are ones in need of treatment for anemia or in need of an increase in hemoglobin. Such subjects may be ones undergoing a treatment for anemia or undergoing a treatment for increasing hemoglobin. These and other suitable subjects are further described herein. The invention contemplates that the decreasing of the platelet count and the treatment for anemia and/or for increasing hemoglobin are simultaneously achieved by the administration of the compound of the invention.

In the methods of the present invention for increasing hemoglobin in a subject, the increase in hemoglobin achieved will depend upon the baseline pre-treatment hemoglobin level and the target hemoglobin range desired. The target hemoglobin range is the maximum desirable level of hemoglobin for the treated subjects. Typically the target hemoglobin range is between 11-13 g/dL, or between 10.5-12 g/dL, or between 10.5-13 g/dL, and generally will not be more than 14 g/dL. Once the target hemoglobin range is achieved, doses of the compound of the invention will be adjusted in order to maintain the hemoglobin level in the treated subject within the target hemoglobin range. In addition, the doses of the compound of the invention administered to the subjects will be adjusted so that the increase in hemoglobin in the treated subjects does not increase by more than 2 g/dL in any 4 week interval.

In the method of the present invention of treating anemia in a subject, determination of the efficacy of the treatment is well within the competence of medical practitioners in the field, and for example can be by measuring of any of a number of well known parameters including, hemoglobin level, hematocrit, CBC, mean corpuscular volume, etc.

The methods of the invention of treating anemia in a subject without significantly increasing the platelet count, of increasing hemoglobin in a subject without significantly increasing the platelet count, of maintaining the platelet count in a subject in need of treatment for anemia, of maintaining the platelet count in a subject in need of an increase in hemoglobin, of decreasing the platelet count in a subject in need of treatment for anemia, of decreasing the platelet count in a subject in need of an increase in hemoglobin of increasing hemoglobin and decreasing the platelet count in a subject with low hemoglobin, and of treating anemia and decreasing the platelet count in a subject with anemia, are accomplished by administering to the subject a therapeutically effective amount of a compound that inhibits HIF prolyl hydroxylase; in particular embodiments the compound is (4-hydroxy-1-methyl-7-phenoxy-isoquinoline-3-carbonyl)-amino]-acetic acid. Preferably the compound is administered orally, one, two, or three times weekly, in a dose of from 0.5 mg/kg to 5.0 mg/kg. Other modes of administration and dosing regimens for use in the methods are described elsewhere herein.

### Subjects

A suitable subject for the methods of the present invention is one in need of an increase in hemoglobin and/or one in need of treatment for anemia. A subject in need of treatment of anemia can be a subject having anemia, or a subject at risk of having anemia. A subject in need of an increase in hemoglobin can be a subject having low hemoglobin, or can be a subject at risk of having low hemoglobin. A subject suitable for treatment using the methods of the present invention is a subject having low hemoglobin, that is, a hemoglobin level below a normal level.

Normal hemoglobin levels for various mammalian species are well known in the art. In particular, for humans, normal hemoglobin levels range from 13 g/dL 18 g/dL for males and 12 g/dL-16 g/dL for females. In particular embodiments, a subject suitable for treatment with the methods of the present invention is a subject having a baseline pre-treatment hemoglobin level below a normal level, such as a human adult having a hemoglobin level below 13 g/dL, below 12 g/dL, below 11.5 g/dL, or below 11.0 g/dL, or below 10.5 g/dL, or below 10 g/dL, or below 9.5 g/dL, or 9.0 g/dL or less, or 8.5 g/dL or less. In other embodiments, a subject suitable for treatment with the methods of the present invention is a human subject having a baseline pre-treatment hemoglobin level of 13 g/dL or less, 12 g/dL or less, 11.5 g/dL or less, or 11.0 g/dL, or less, or 10.5 g/dL or less, or 10 g/dL or less, or 9.5 g/dL or less, or 9.0 g/dL or less, or 8.5 g/dL or less.

In some cases a suitable subject will be a subject having, or at risk of having, anemia. A subject in need of treatment for anemia is a subject having anemia. Such anemic subjects can be readily identified by competent medical practitioners and/or by using routine conventional testing for anemia. A subject in need of treatment for anemia may also be a subject at risk of having anemia. Such an at risk subject may be a subject having a condition or disorder that is associated with, or increases the occurrence of, anemia in the subject, for example, acute or chronic kidney disease, polycystic kidney disease, end stage renal disorder, cancer, chemotherapy treatments, ulcers, diabetes, immunosuppressive disease, infection, inflammation, blood loss (for example, blood less associated with bleeding disorders, trauma, injury, or surgery), etc. In yet another aspect, the anemia is associated with a procedure or treatment selected from the group consisting of radiation therapy, chemotherapy, dialysis, and surgery. In specific embodiments, the subject in need of treatment for anemia may be an HIV-infected anemic subject being treated with zidovudine or other reverse transcriptase inhibitors, or an anemic cancer patient receiving cyclic cisplatin- or non-cisplatin-containing chemotherapy. In particular embodiments, the subjects at risk of having anemia may be a subject scheduled to undergo elective, noncardiac, nonvascular surgery, thereby reducing the need for allogenic blood transfusions or to facilitate banking of blood prior to surgery. Subjects at risk of having anemia may also be at risk for having low hemoglobin.

In some embodiments the subject will have a baseline platelet count in the normal range. In some embodiments the subject will have a baseline platelet count in the high normal range. In some embodiments the subject will have a baseline platelet count in the low normal range. In some embodiments the subject will have a baseline platelet count of greater than 150,000/ul, greater than 200,000/ul, greater than 220,000/ul, greater than 240,000/ul, greater than 260,000/ul, or greater than 280,000/ul. In some embodiments the subject will have a baseline platelet count of between 150,000/ul and 300,000/ul. In some embodiments the subject will have a baseline platelet count of greater than 300,000/ul, greater than 320,000/ul, greater than 340,000/ul, greater than 360,000/ul, or greater than 380,000/ul. In some embodiments the subject will have a baseline platelet count of between 300,000/ul and 400,000/ul.

The subject for the methods of the present invention is an animal, preferably a mammal (e.g., a dog, a cat, a horse, a monkey, a human, etc.). The preferred subject is a human subject.

Methods for measuring the hemoglobin level in a subject are routine hematological practices. Various methods for measuring the erythropoietin level in a subject are well-known and available to one of ordinary skill in the art. For example, the erythropoietin level in a subject can be measured using a commercially-available ELISA.

### Compounds

Compounds for use in the methods or medicaments provided herein are inhibitors of hypoxia-inducible factor (HIF) prolyl hydroxylase enzymes. In certain embodiments, the compound that inhibits HIF prolyl hydroxylase enzyme activity for use in the claimed methods is [(4-hydroxy-1-methyl-7-phenoxy-isoquinoline-3-carbonyl)-amino]-acetic acid (Compound A). Methods for making Compound A are described in detail in, inter alia, U.S. Pat. No. 7,323,475, and U.S. Pat. No. 8,017,475, which patents are incorporated herein by reference in their entireties.

A compound that inhibits the activity of HIF prolyl hydroxylase enzyme refers to any compound that reduces or otherwise modulates the activity of at least one HIF prolyl hydroxylase enzyme. The term “HIF prolyl hydroxylase,” as used herein, refers to any enzyme that is capable of hydroxylating a proline residue within an alpha subunit of HIF. Such HIF prolyl hydroxylases include protein members of the EGL-9 (EGLN) 2-oxoglutarate- and iron-dependent dioxygenase family described by Taylor (2001) Gene 275:125-132; and characterized by Aravind and Koonin (2001) Genome Biol 2:RESEARCH0007; Epstein et al. (2001) Cell 107:43-54; and Bruick and McKnight (2001) Science 294:1337-1340.

Methods for determining whether a compound inhibits HIF prolyl hydroxylase are well known in the art and a number of techniques are described herein

Functionally, HIF prolyl hydroxylase inhibitors for use in the methods of the present invention are defined by their ability to inhibit an activity of a 2-oxoglutarate dioxygenase enzyme, wherein the enzyme has specific activity toward hypoxia inducible factor. Such compounds are often referred to as prolyl hydroxylase inhibitors or “PHI”s. Preferably, the PHIs for use in the invention are small molecule compounds. A compound that inhibits the activity of a HIF prolyl hydroxylase enzyme refers to any compound that reduces or otherwise modulates the activity of at least one HIF prolyl hydroxylase enzyme. A compound may additionally show inhibitory activity toward one or more other 2-oxoglutarate- and iron-dependent dioxygenase enzymes, e.g. factor inhibiting HIF (FIH; GenBank Accession No. AAL27308), procollagen prolyl 4-hydroxylase (CP4H), etc.

In particular embodiments, compounds used in the present methods and medicaments provided herein are structural mimetics of 2-oxoglutarate, wherein the compound inhibits the target HIF prolyl hydroxylase enzyme competitively with respect to 2-oxoglutarate and noncompetitively with respect to iron. PHIs are typically heterocyclic carboxamide compounds, especially heterocyclic carbonyl glycine derivatives, and may be, for example, pyridine, pyrimidine, pyridazine, naphthyridine, pyrrolopyridine, thiazolopyridine, isothiazolopyridine, quinoline, isoquinoline, einnoline, beta-carboline, quinolone, thienopyridine, chromene, or thiochromene carboxamides. Compounds that inhibit HIF prolyl hydroxylase are known in the art and are described in, inter alia, U.S. Pat. Nos. 5,658,933; 5,620,995; 5,719,164; 5,726,305; 6,093,730; 7,323,475; U.S. application Ser. No. 12/544,861; U.S. Patent Application Publication Nos. 2006/0199836; 2007/0298104; 2008/0004309; and PCT publication Nos. WO2009/073669; WO2009/089547; WO2009/100250; U.S. Patent Application Publication 2003/0176317, U.S. Patent Application Publication 2003/0153503, U.S. Pat. No. 7,323,475, U.S. Patent Application Publication 2006/0199836, U.S. Pat. No. 7,928,120, U.S. Pat. No. 7,696,223, U.S. Patent Application Publication 2010/0303928, U.S. Patent Application Publication 2010/0330199, U.S. Patent Application Publication 2010/0331400, U.S. Patent Application Publication 2010/0047367, PCT Application No. PCT/US2009/064065, U.S. Pat. No. 7,897,612, U.S. Pat. No. 7,608,621, U.S. Pat. No. 7,728,130, U.S. Pat. No. 7,635,715, U.S. Pat. No. 7,569,726, U.S. Pat. No. 7,811,595. The foregoing patents and patent applications are incorporated in their entireties herein. Other prolyl hydroxylase inhibitors are well known and have been described in the art.

Methods of determining if any particular compound inhibits HIF prolyl hydroxylase are well known, for example, the methods described in U.S. Pat. No. 7,323,475. The IC₅₀ for Compound A for each of the HIF prolyl hydroxylase enzymes can be determined in the assays described herein. For Compound A, the IC₅₀s for PHD1, PHD2, and PHD3 are very similar and are all in the micromolar range from about 0.2 to 2 μM.

In certain embodiments, compounds used in the methods of the invention are selected from a compound of the formula (I)

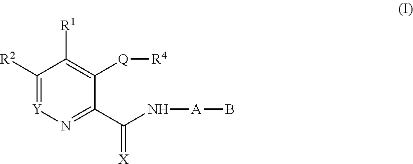

wherein
A is 1,2-arylidene, 1,3-arylidene, 1,4-arylidene; or (C₁-C₄)-alkylene, optionally substituted by one or two halogen, cyano, nitro, trifluoromethyl, (C₁-C₆)-alkyl, (C₁-C₆)-hydroxyalkyl, (C₁-C₆)-alkoxy, —O—[CH₂]ₓ—C_{f}H_{(2f+1-g)} Hal_{g}, (C₁-C₆)-fluoroalkoxy, (C₁-C₈)-fluoroalkenyloxy, (C₁-C₈)-fluoroalkynyloxy, —OCF₂Cl, —O—CF₂—CHFCl; (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, phenyl, benzyl, phenoxy, benzyloxy, anilino, N-methylanilino, phenylmercapto, phenylsulfonyl, phenylsulfinyl, sulfamoyl, N—(C₁-C₄)-alkylsulfamoyl, N,N-di-(C₁-C₄)-alkylsulfamoyl; or by a substituted (C₆-C₁₂)-aryloxy, (C₇-C₁₁)-aralkyloxy, (C₆-C₁₂)-aryl, (C₇-C₁₁)-aralkyl radical, which carries in the aryl moiety one to five identical or different substituents selected from halogen, cyano, nitro, trifluoromethyl, (C₁-C₆)-alkyl, (C₁-C₆)-alkoxy, —O—[CH₂]ₓ—C_{f}H_{(2f+1-g)} Hal_{g}, —OCF₂Cl, —O—CF₂—CHFCl, (C₁-C₆)-alkylmercapto, (C₁-C₆)-alkylsulfinyl, (C₁-C₆)-alkylsulfonyl, (C₁-C₆)-alkylcarbonyl, alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, sulfamoyl, N—(C₁-C₄)-alkylsulfamoyl, N,N-di-(C₁-C₄)-alkylsulfamoyl; or wherein A is —CR⁵R⁶ and R⁵ and R⁶ are each independently selected from hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H, —NHSO₂CF₃, tetrazolyl, imidazolyl, 3-hydroxyisoxazolyl, CONHCOR′″, —CONHSOR′″, CONHSO₂R′″, where R′″ is aryl, heteroaryl, (C₃-C₇)-cycloalkyl, or (C₁-C₄)-alkyl, optionally monosubstituted by (C₆-C₁₂)-aryl, heteroaryl, OH, SH, (C₁-C₄)-alkyl, (C₁-C₄)-alkoxy, (C₁-C₄)-thioalkyl, (C₁-C₄)-sulfinyl, (C₁-C₄)-sulfonyl, CF₃, Cl, Br, F, I, NO2, —COOH, (C₂-C₅)-alkoxycarbonyl, NH₂, mono-(C₁-C₄-alkyl)-amino, di-(C₁-C₄-alkyl)-amino, or (C₁-C₄)-perfluoroalkyl; or wherein 8 is a CO₂-G carboxyl radical, where G is a radical of an alcohol G-OH in which G is selected from (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical, where the alkenyl, cycloalkenyl, alkynyl, and alkenynyl radicals contain one or more multiple bonds; (C₆-C₁₆)-carbocyclic aryl radical, (C₇-C₁₆)-carbocyclic aralkyl radical, heteroaryl radical, or heteroaralkyl radical, wherein a heteroaryl radical or heteroaryl moiety of a heteroaralkyl radical contains 5 or 6 ring atoms; and wherein radicals defined for G are substituted by one or more hydroxyl, halogen, cyano, trifluoromethyl, nitro, carboxyl, (C₁-C₁₂)-alkyl, (C₃-C₈)-cycloalkyl, (C₅-C₈)-cycloalkenyl, (C₆-C₁₂)-aryl, (C₇-C₁₆)-aralkyl, (C₂-C₁₂)-alkenyl, (C₂-C₁₂)-alkynyl, (C₁-C₁₂)-alkoxy, (C₁-C₁₂)-alkoxy-(C₁-C₁₂)-alkyl, (C₁-C₁₂)-alkoxy-(C₁-C₂)-alkoxy, (C₆-C₁₂)-aryloxy, (C₇-C₁₆)-aralkyloxy, (C₁-C₈)-hydroxyalkyl, —O—[CH₂]ₓ—C_{f}H_{(2f+1-g)}—F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl cinnamoyl, (C₂-C₁₂)-alkenylcarbonyl, (C₂-C₁₂)-alkynylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, acyloxy, (C₁-C₁₂)-alkoxycarbanyloxy, (C₁-C₁₂)-alkoxy-(C₁-C₁₂)-alkoxycarbonyloxy, (C₆-C₁₂)-aryl oxycarbonyloxy, (C₇-C₁₆) aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N.N-di(C₁-C₁₂)-alkylcarbamoyl, N—(C₃-C₈)-cycloalkylcarbamoyl, N—(C₆-C₁₆)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₀)-alkoxy-(C₁-C₁₀)-allyl)-carbamoyl, N—((C₆-C₁₂)-aryloxy-(C₁-C₁₀)alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₅-C₁₆)-aryloxy-(C₁-C₁₀)-alkyl)-carbamoyl, N—(C₁-C₁₀)-alkyl-N—(C₇-C₁₆)-aralkyloxy-(C₁-C₁₀)-alkyl)-carbamoyl, carbamoyloxy, N—(C₁-C₁₂)-alkylcarbamoyloxy, N.N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N(C₁-C₁₀)-alkyl-N—(C₁-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₂-C₁₂)-alkenylamino, (C₂-C₁₂)-alkynylamino, N—(C₆-C₁₂)-arylamino, N—(C-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂) arylcarbonylamino, (C₇-C₁₆)-aralkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)alkyl, (C₆-C₁₂)-arylcarbonylamino-(C₁-C₈)-alkyl, (C₇-C₁₂)-aralkylcarbonylamino(C₁-C₈)-alkyl, amino-(C₁-C₁₀)-alkyl, N—(C₁-C₁₀) alkylamino-(C₁-C₁₀)-alkyl. N,N-di-(C₁-C₁₀)-alkylamino-(C₁-C₁₀)-alkyl, (C₃-C₈)cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di(C₁-C₁₀-alkylsulfamoyl, (C₃-C₈)-cycloalkylsulfamoyl, N—(C₆-C₁₂)-alkylsulfamoyl, N—(C₇-C₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁)-alkylsulfonamido, (C₇-C₁₆)-aralkylsulfonamido, or N—((C₁-C₁₀)-alkyl-(C₇-C₁₆)-aralkylsulfonamido; wherein radicals which are aryl or contain an aryl moiety, may be substituted on the aryl by one to five identical or different hydroxyl, halogen, cyano, trifluoromethyl, nitro, carboxyl, (C₁-C₁₂)-alkyl, (C₃-C₈)-cycloalkyl, (C₆-C₁₂)-aryl, (C₇-C₁₆)-aralkyl, (C₁-C₁₂)-alkoxy, (C₁-C₁₂)-alkoxy-(C₁-C₁ (C₁-C₁₂)-alkoxy-(C₁-C₁₂)alkoxy, (C₆-C₁₂)-aryloxy, (C₇-C₁₆)-aralkyloxy, (C₁-C₈)-hydroxyalkyl, (C₁-C₁₂)-alkylcarbonyl, (C₃-C₈)-cycloalkyl-carbonyl, (C₆-C₁₂)-arylcarbonyl, (C₇-C₁₆) aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N.N-di-(C₁-C₁₂)-alkylcabamoyl, N—(C₃-C₈)-cycloalkylcarbamoyl, N—(C₆-C₁₂)-arylcarbamoyl, N—(C₇-C₁₆)-aralkylcarbamoyl, N—(C₁-C₁₀)-alkyl-N—(C₆-C₁₂)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryl oxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—(C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy; —(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkylaralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-alkylcarbonylamino, (C₁-C₁₂)-alkylcarbonyl-N—(C₁-C₁₀)-alkylamino, (C₃-C₈)-cycloalkyl/carbonyl-N—(C₁-C₁₀)-alkylamino, (C₆-C₁₂)-arylcarbonyl-N—(C₁-C₁₀)-alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)-alkyl, (C₆-C₁₂)-arylcarbonylamino-(C₁-C₈)-alkyl, (C₇-C₁₆)-aralkylcarbonylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, or (C₇-C₁₆)-aralkylsulfonyl;
X is O or S;
Q is O, S, NR′, or a bond;
where, if Q is a bond, R⁴ is halogen, nitrite, or trifluoromethyl; or where, if Q is O, S, or NR′, R⁴ is hydrogen, (C₁-C₁₀)-alkyl radical, (C₂-C₁₀)-alkenyl radical, (C₂-C₁₀)-alkynyl radical, wherein alkenyl or alkynyl radical contains one or two CC multiple bonds; unsubstituted fluoroalkyl radical of the formula —[CH₂]ₓ—C_{f}H_{(2f+1-g)}—F_{g}, (C₁-C₈)-alkoxy-(C₁-C₆)-alkyl radical, (C₁-C₆)-alkoxy-(C₁-C₄)-alkoxy-(C₁-C₄)-alkyl radical, aryl radical, heteroaryl radical, (C₇-C₁₁)-aralkyl radical, or a radical of the formula Z

—[CH₂]ᵥ—[O]_{w}—[CH₂]ₜ-E (Z)
where
   E is a heteroaryl radical, a (C₃-C₈)-cycloalkyl radical, or a phenyl radical of the formula F

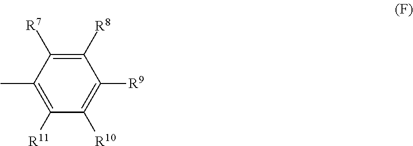

v is 0-6,
w is 0 or 1
t is 0-3, and
R⁷, R⁸, R⁹, R¹⁰, and R¹¹ are identical or different and are hydrogen, halogen, cyano, nitro, trifluoromethyl, (C₁-C₆)-alkyl, (C₃-C₈)-cycloalkyl, (C₁-C₆)-alkoxy, —O—[CH₂]ₓ—C_{f}H_{(2f+1-g)}—F_{g}, —OCF₂—Cl, —O—CF₂—CHFCl, (C₁-C₆)-alkylmercapto, (C₁-C₆)-hydroxyalkyl, (C₁-C₆)-alkoxy-(C₁-C₆)-alkoxy, (C₁-C₆)-alkoxy-(C₁-C₆)-alkyl, (C₁-C₆)-alkylsulfinyl, (C₁-C₆)-alkylsulfonyl, (C₁-C₆)-alkylcarbonyl, (C₁-C₈)-alkoxycarbonyl, carbamoyl, N—(C₁-C₈)-alkylcarbamoyl, N,N-di-(C₁-C₈)-alkylcarbamoyl, or (C₇-C₁₁)-aralkylcarbamoyl, optionally substituted by fluorine, chlorine, bromine, trifluoromethyl, (C₁-C₆)-alkoxy, N—(C₃-C₈)-cycloalkylcarbamoyl, N—(C₃-C₈)-cycloalkyl-(C₁-C₄)-alkylcarbamoyl, (C₁-C₆)-alkylcarbonyloxy, phenyl, benzyl, phenoxy, benzyloxy, NR^{Y}R^{Z} wherein R^{y} and R^{z} are independently selected from hydrogen, (C₁-C₁₂)-alkyl, (C₁-C₈)-alkoxy-(C₁-C₈)-alkyl, (C₇-C₁₂)-aralkoxy-(C₁-C₈)-alkyl, (C₆-C₁₂)-aryl oxy-(C₁-C₈)-alkyl, (C₃-C₁₀)-cycloalkyl, (C₃-C₁₂)-alkenyl, (C₃-C₁₂)-alkynyl, (C₆-C₁₂)-aryl, (C₇-C₁₁)-aralkyl, (C₁-C₁₂)-alkoxy, (C₇-C₁₂)aralkoxy, (C₁-C₁₂)-alkylcarbonyl, (C₃-C₈)-cycloalkylcarbonyl, (C₆-C₁₂) arylcarbonyl, (C₇-C₁₆)-aralkylcarbonyl; or further wherein R^{y} and R^{z} together are —[CH2]ₕ, in which a CH₂ group can be replaced by O, S, N—(C₁-C₄)-alkylcarbonylimino, or N—(C₁-C₄)-alkoxycarbonylimino; phenylmercapto, phenylsulfonyl, phenylsulfinyl, sulfamoyl, N—(C₁-C₈)-alkylsulfamoyl, or N,N-di-(C₁-C₈)-alkylsulfamoyl; or alternatively R⁷ and R⁸, R⁸ and R⁹, R⁹ and R¹⁰, or R¹⁰ and R¹¹, together are area chain selected from —[CH₂]ₙ— or —CH═CH—CH═CH—, where a CH₂ group of the chain is optionally replaced by O, S, SO, SO₂, or NR^{Y}; and n is 3, 4, or 5; and if E is a heteroaryl radical, said radical can carry 1-3 substituents selected from those defined for R⁷-R¹¹, or if E is a cycloalkyl radical, the radical can carry one substituent selected from those defined for R⁷-R¹¹;
or where, if Q is NR′, R⁴ is alternatively R″, where R′ and R″ are identical or different and are hydrogen, (C₆-C₁₂)-aryl, (C₇-C₁₁)-aralkyl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkylcarbonyl, optionally substituted (C₇-C₁₆)-aralkylcarbonyl, or optionally substituted C₆-C₁₂)-arylcarbonyl; or R′ and R″ together are —[CH₂]ₕ, in which a CH₂ group can be replaced by O, S, N-acylimino, or N—(C₁-C₁₀)-alkoxycarbonylimino, and h is 3 to 7;
Y is N or CR³;
R¹, R² and R³ are identical or different and are hydrogen, hydroxyl, halogen, cyano, trifluoromethyl, nitro, carboxyl, (C₁-C₂₀)-alkyl, (C₃-C₈)-cycloalkyl, (C₃-C₈)cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₁-C₂₀)-alkoxy-(C₁-C₁₂)-alkyl, (C₁-C₁₂)alkoxy-(C₁-C₁₂)-alkoxy, (C₁-C₁₂)-alkoxy-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₁₆)-hydroxyalkyl, (C₆-C₁₆)-aryloxy-(C₁-C₈)-alkyl, (C₇-C₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, (C₂-C₂₀)-alkenyloxy-(C₁-C₆)-alkyl, (C₂-C₂₀)-alkynyloxy-(C₁-C₅)-alkyl, retinyloxy-(C₁-C₆)-alkyl, —O—[CH₂]ₓ—CfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₂-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N—(C₁-C₆)-alkyl-N—((C₃-C₈)-cycloalkyl-(C₁-C₆)-alkyl)-carbamoyl, N-(+)-dehydroabietylcarbamoyl, N—(C₁-C₆)-alkyl-N-(±)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₈)-alkoxy-(C₁-C₁₀)-alkyl)-carbamoyl, N—((C₆-C₁₆)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀-alkyl)-carbamoyl; CON(CH₂)ₕ, in which a CH₂ group can be replaced by O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; a carbamoyl radical of the formula R

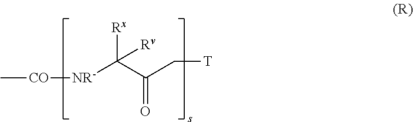

in which
R^{x} and R^{v} are each independently selected from hydrogen, (C₁-C₆)-alkyl, (C₃-C₇)-cycloalkyl, aryl, or the substituent of an α-carbon of an α-amino acid, to which the L- and D-amino acids belong,
s is 1-5,
T is OH, or NR*R**, and R*, R** and R*** are identical or different and are selected from hydrogen, (C₆-C₁₂)-aryl, (C₇-C₁₁)-aralkyl, (C₁-C₈)-alkyl, (C₃-C₈)-cycloalkyl, (+)-dehydroabietyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, optionally substituted (C₆-C₁₂)-aroyl; or R* and R** together are —[CH₂]ₕ, in which a CH₂ group can be replaced by O, S, SO, SO₂, N-acylamino, N—(C₁-C₁₀)-alkoxycarbonylimino, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7;
carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy. N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₁-C₁₂)-alkynylamino, N—(C₆-C₁₂)^{arylamino, N—(C}₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di(C₁-C₁₀)-alkylamino-(C₁-C₁₀)-alkyl, (C₃-C₈)-cycloalkylamino(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, (C₁-C₁₂)-alkylmercapto-(C₁-C₆)-alkyl, (C₁-C₁₂)-alkylsulfinyl-(C₁-C₆)-alkyl, (C₁-C₁₂)-alkylsulfonyl-(C₁-C₆)-alkyl, (C₆-C₁₂)-arylmercapto-(C₁-C₆)-alkyl, (C₆-C₁₂)-arylsulfinyl-(C₁-C₆)-alkyl, (C₆-C₁₂)-arylsulfonyl-(C₁-C₆)-alkyl, (C₇-C₁₆)-aralkylmercapto-(C₁-C₆)-alkyl, (C₇-C₁₆)-aralkylsulfinyl-(C₁-C₆)-alkyl, (C₇-C₁₆)-aralkylsulfonyl-(C₁-C₆)-alkyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N-(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, and N—((C₁-C₁₀)-alkyl-(C₇-C₁₆)-aralkylsulfonamido; were an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkyl-(C₁-C₁₂)-all, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₅)-alkenyloxy, (C₁-C₁₂)-alkoxy-(C₁-C₁₂)-alkyl, (C₁-C₁₂)-alkoxy-(C₁-C₁₂)-alkoxy, (C₁-C₁₂)-alkoxy(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₈)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, —O—[CH₂]ₓ—C_{f}H_{(2f+1-g)}—F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂) alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)carbamoyl, N—(C₁-C₆)-alkyl-N—((C₃-C₈)-cycloalkyl-(C₁-C₆)-alkyl)carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₆)-alkoxy-(C₁-C₁₀)-alkyl)carbamoyl, N—((C₆-C₁₆)-aryloxy-(C₁-C₁₀)-alkyl)carbamoyl, N—((C₇-C₁₆)-aralkyloxy-(C₁-C₁₀)-alkyl)carbamoyl, N—(C₁-C₁₀)-alkyl-N—((C₁-C₁₀)-alkoxy-(C₁-C₁₀)-alkyl)carbamoyl, N—(C₁-C₁₀)-alkyl-N—((C₆-C₁₂)-aryloxy-(C₁-C₁₀)-alkyl)carbamoyl, N—(C₁-C₁₀)-alkyl-N—((C₇-C₁₆)-aralkyloxy-(C₁-C₁₀)-alkyl)-carbamoyl, CON(CH₂)ₕ, in which a CH₂ group can be replaced by, O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₆)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)carbamoyloxy, N—((C₆-C₁₂)-aryloxy-(C₁-C₁₀)-alkyl)carbamoyloxy, N—((C₇-C₁₆)-aralkyloxy-(C₁-C₁₀-alkyl)carbamoyloxy, N—(C₁-C₁₀)-alkyl-N—((C₁-C₁₀)-alkoxy-(C₁-C₁₀)-alkyl)carbamoyloxy, N—(C₁-C₁₀)-alkyl-N—((C₆-C₁₂)-aryloxy-(C₁-C₁₀)-alkyl)carbamoyloxy, N—(C₁-C₁₀)-alkyl-N—((C₇-C₁₆)-aralkyloxy-(C₁-C₁₀)-alkyl)carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino. N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)alkoxy-N—(C₁-C₁₀)-alkylamino, (C₁-C₁₂)-alkanoylamino, (C₃-C₈)-cycloalkanoylamino, (C₆-C₁₂)aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₅)-aralkanoylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino-(C₁-C₁₀-alkyl, (C₁-C₁₂)-alkylmercapto (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₆)-arylsulfonyl, (C₇-C₁₆)-aralkylmercapto, (C₇-C₁₆)-aralkylsulfinyl, or (C₇-C₁₆)-aralkylsulfonyl;
or wherein R¹ and R², or R² and R³ form a chain [CH₂]ₒ which is saturated or unsaturated by a C═C double bond, in which 1 or 2 CH₂ groups are optionally replaced by O, S, SO, SO₂, or NR′, and R′ is hydrogen, (C₆-C₁₂)-aryl, (C₁-C₈)-alkyl, (C₁-C₈)-alkoxy-(C₁-C₈)-alkyl, (C₇-C₁₂)aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, or optionally substituted (C₆-C₁₂)-aroyl; and o is 3, 4 or 5;
or wherein the radicals R¹ and R², or R² and R³, together with the pyridine or pyridazine carrying them, form a 5,6,7,8-tetrahydroisoquinoline ring, a 5,6,7,8-tetrahydroquinoline ring, or a 5,6,7,8-tetrahydrocinnoline ring;
or wherein R¹ and R², or R² and R³ form a carbocyclic or heterocyclic 5- or 6-membered aromatic ring;
or where R¹ and R², or R² and R³, together with the pyridine or pyridazine carrying them, form an optionally substituted heterocyclic ring systems selected from thienopyridines, furanopyridines, pyridopyridines, pyrimidinopyridines, imidazopyridines, thiazolopyridines, oxazolopyridines, quinoline, isoquinoline, and cinnoline; where quinoline, isoquinoline or cinnoline preferably satisfy the formulae Ia, Ib and Ic:

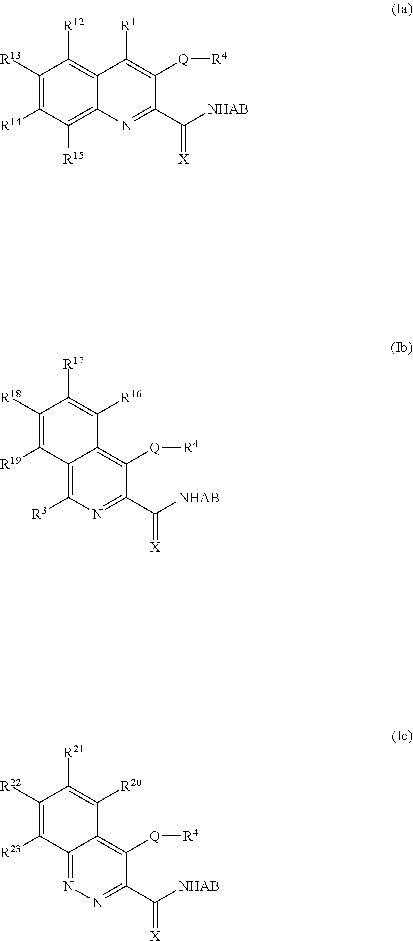

and the substituents R¹² to R²³ in each case independently of each other have the meaning of R¹, R² and R³; or wherein the radicals R¹ and R², together with the pyridine carrying them, form a compound of Formula Id:

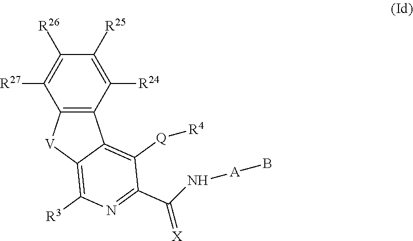

where V is S, O, or NR^{k}, and R^{k} is selected from hydrogen, (C₁-C₆)-alkyl, aryl, or benzyl; where an aryl radical may be optionally substituted by 1 to 5 substituents as defined above; and
R²⁴, R²⁵, R²⁶, and R²⁷ in each case independently of each other have the meaning of R¹, R² and R³;
f is 1 to 8;
g is 0 or 1 to (2f+1);
x is 0 to 3; and
h is 3 to 7;
including the physiologically active salts and prodrugs derived therefrom.

Exemplary compounds according to Formula (I) are described in European Patent Nos. EP0650960 and EP0650961. All compounds listed in EP0650960 and EP0650961, in particular, those listed in the compound claims and the final products of the working examples, are hereby incorporated into the present application by reference herein.

Additionally, exemplary compounds according to Formula (I) are described in U.S. Pat. No. 5,658,933. All compounds listed in U.S. Pat. No. 5,658,933, in particular, those listed in the compound claims and the final products of the working examples, are hereby incorporated into the present application by reference herein.

Additional compounds according to Formula (I) are substituted heterocyclic carboxyamides described in U.S. Pat. No. 5,620,995; 3-hydroxypyridine-2-carboxamidoesters described in U.S. Pat. No. 6,020,350; sulfonamidocarbonylpyridine-2-carboxamides described in U.S. Pat. No. 5,607,954°, and sulfonamidocarbonyl-pyridine-2-carboxamides and sulfonamidocarbonyl-pyridine-2-carboxamide esters described in U.S. Pat. Nos. 5,610,172 and 5,620,996. All compounds listed in these patents, in particular, those compounds listed in the compound claims and the final products of the working examples, are hereby incorporated into the present application by reference herein.

Exemplary compounds according to Formula (Ia) are described in U.S. Pat. Nos. 5,719,164 and 5,726,305. All compounds listed in the foregoing patents, in particular, those listed in the compound claims and the final products of the working examples, are hereby incorporated into the present application by reference herein.

Exemplary compounds according to Formula (Ib) are described in U.S. Pat. No. 6,093,730. All compounds listed in U.S. Pat. No. 6,093,730, in particular, those listed in the compound claims and the final products of the working examples, are hereby incorporated into the present application by reference herein.

In particular embodiments, the compounds used in the methods and medicaments for treating anemia in a subject having anemia, or at risk of having anemia, or for increasing hemoglobin levels in a subject in need thereof, are structural mimetics of 2-oxoglutarate, which may inhibit the target HIF prolyl hydroxylase enzyme competitively with respect to 2-oxoglutarate and noncompetitively with respect to iron. In another embodiment, compounds for use in the present methods and medicaments are heterocyclic carbonyl glycines of formula A:

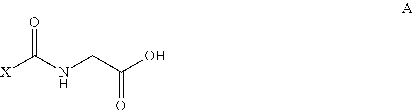

wherein X is an optionally substituted heterocyclic moiety. Such prolyl hydroyxlase inhibitors (PHIs) include, but are not limited to, variously substituted 3-hydroxy-pyridine-2-carbonyl-glycines, 4-hydroxy-pyridazine-3-carbonyl-glycines, 3-hydroxy-quinoline-2-carbonyl-glycines, 4-hydroxy-2-oxo-1,2-dihydro-quinoline-3-carbonyl-glycines, 4-hydroxy-2-oxo-1,2-dihydro-naphthyridine-3-carbonyl-glycines, 8-hydroxy-6-oxo-4,6-dihydro-pyridopyrazine-7-carbonyl-glycines, 4-hydroxy-isoquinoline-3-carbonyl-glycines, 4-hydroxy-cinnoline-3-carbonyl-glycines, 7-hydroxy-thienopyridine-6-carbonyl-glycines, 4-hydroxy-thienopyridine-5-carbonyl-glycines, 7-hydroxy-thiazolopyridine-6-carbonyl-glycines, 4-hydroxy-thiazolopyridine-5-carbonyl-glycines, 7-hydroxy-pyrrolopyridine-6-carbonyl-glycines, 4-hydroxy-pyrrolopyridine-5-carbonyl-glycines, etc.

The term “alkyl” refers to saturated monovalent hydrocarbyl groups and is exemplified by groups such as methyl, ethyl, n-propyl, iso-propyl, and the like. An alkyl substituted with one or more alkyl may include, but is not limited to, n-butyl, t-butyl, n-pentyl, 2-methyl-pentyl, 1-ethyl-2-methyl-pentyl, and the like. An alkyl substituted by an aryl may include, but is not limited to, benzyl, 1-naphthalen-2-yl-ethyl, and the like.

The term “alkoxy” refers to the group “alkyl-O—” and includes, by way of example, methoxy, ethoxy, n-propoxy, iso-propoxy, and the like.

The term “aryl” refers to a monovalent aromatic carbocyclic group having a single ring or multiple condensed rings and includes, by way of example, phenyl, naphthyl, and the like.

The term “aryloxy” refers to the group aryl-O— and includes, by way of example, phenoxy, naphthoxy, and the like.

The term “cyano” refers to the group —CN.

The term “halo” or “halogen” refers to fluoro, chloro, bromo, and iodo.

Suitable compounds for use in the methods and medicaments of the invention may be identified using any conventionally known methods. Suitable assay methods are well known in the art. For example, compounds may be tested for their ability to inhibit the activity of a HIF prolyl hydroxylase in an enzyme assay as described elsewhere herein. Compounds are combined with radiolabeled α-ketoglutarate, a hydroxylatable HIFα peptide, and a HIF prolyl hydroxylase, e.g., EGLN3 under conditions where, in the absence of compound, the HIF prolyl hydroxylase is capable of hydroxylating the HIFα peptide and converting the α-ketoglutarate to succinate and carbon dioxide; and levels of liberated carbon dioxide are measured, wherein a reduction in the amount of liberated carbon dioxide in the presence of compound identifies an inhibitor of HIF prolyl hydroxylase. Methods of determining if any particular compound inhibits HIF prolyl hydroxylase are well known, for example, the methods described in U.S. Pat. No. 7,323,475. The IC₅₀ for Compound A for each of the HIF prolyl hydroxylase enzymes can be determined in the assays described above.

### Methods for Identifying Compounds

A compound suitable for use in the method, or for manufacture of a medicament, of the invention is one that inhibits HIF hydroxylase activity. Methods for identifying compounds suitable for use in the method, or for manufacture of a medicament, of the invention are also provided. Assays for hydroxylase activity are standard in the art. Such assays can directly or indirectly measure hydroxylase activity. For example, an assay can measure hydroxylated residues, e.g., proline, etc., present in the enzyme substrate, e.g., a target protein, a synthetic peptide mimetic, or a fragment thereof. (See, e.g., Palmerini et al. (1985) J Chromatogr 339:285-292.) A reduction in hydroxylated residue, e.g., proline, in the presence of a compound is indicative of a compound that inhibits hydroxylase activity. Alternatively, assays can measure other products of the hydroxylation reaction, e.g., formation of succinate from 2-oxoglutarate. (See, e.g., Cunliffe et al. (1986) Biochem J 240:617-619.) Kaule and Gunzler (1990; Anal Biochem 184:291-297) describe an exemplary procedure that measures production of succinate from 2-oxoglutarate.

Procedures such as those described above can be used to identify compounds that modulate HIF hydroxylase activity. Target protein may include HIFα or a fragment thereof, e.g., HIF(556-575). Enzyme may include, e.g., HIF prolyl hydroxylase (see, e.g., GenBank Accession No. AAG33965, etc.) or HIF asparaginyl hydroxylase (see, e.g., GenBank. Accession No. AAL27308, etc.), obtained from any source. Enzyme may also be present in a crude cell lysate or in a partially purified form. For example, procedures that measure HIF hydroxylase activity are described in Ivan et al. (2001, Science 292:464-468; and 2002. Proc Natl Acad Sci USA 99:13459-13464) and Hirsila et al. (2003, J Biol Chem 278:30772-30780); additional methods are described in International Publication No. WO 03/049686. Measuring and comparing enzyme activity in the absence and presence of the compound will identify compounds that inhibit hydroxylation of HIFα.

In certain aspects, a suitable compound is one that stabilizes HIFα. Compounds that inhibit HIF prolyl hydroxylase prevent or reduce the hydroxylation of the HIFα subunit of the HIF protein. This lack of hydroxylated proline leads to the stabilization (often referred to as activation) of HIF. Determination of the stabilization of HIF by a compound can be used as an indirect measurer of the ability of the compound to inhibit HIF prolyl hydroxylase. The ability of a compound to stabilize or activate HIFα can be measured, for example, by direct measurement of HIFα in a sample, indirect measurement of HIFα, e.g., by measuring a decrease in HIFα associated with the von Hippel Lindau protein (see, e.g., International Publication No. WO 2000/69908), or activation of HIF responsive target genes or reporter constructs (see, e.g., U.S. Pat. No. 5,942,434). Measuring and comparing levels of HIF and/or HIF-responsive target proteins in the absence and presence of the compound will identify compounds that stabilize HIFα and/or activate HIF. Suitable compounds for use in the present methods may be identified and characterized using the assay described in International Publication No. WO 2005/118836, or in Example 10 of International Publication No. WO 2003/049686, both of which are incorporated herein by reference in their entirety. Compounds identifiable by these assays are specifically envisaged for use in the present invention.

### Pharmaceutical Formulations and Routes of Administration

The compositions and compounds suitable for use in the method, or for manufacture of a medicament, of the present invention can be delivered directly or in pharmaceutical compositions containing excipients, as is well known in the art.

A therapeutically effective amount, e.g., dose, of compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences, supra; and Hardman, Limbird, and Gilman, eds. (2001) The Pharmacological Basis of Therapeutics, supra.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

In preferred embodiments, for use in the method of the invention the compounds of the present invention are administered orally.

Pharmaceutical dosage forms of a suitable compound for use in the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc, 2002.)

Pharmaceutical dosage forms of a compound for use in the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions for use in the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof (e.g., magnesium stearate), sugars (i.e. dextrose, sucrose, lactose, etc.), croscarmellose sodium, talc, tragacanth mucilage, vegetable oils (hydrogenated), microcrystalline cellulose, and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro)-emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as polylactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

For composition useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, i.e., minimal effective concentration MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

In some embodiments of the present invention, therapeutically effective doses for compounds for use in the invention include doses of 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, or 30 mg/kg, and may include doses between these values, for example 1.5 mg/kg or 0.75 mg/kg. For administration in the methods of the present invention for treating anemia, or for increasing hemoglobin, the doses may be adjusted during treatment to maintain a hemoglobin level in the subject within a target range. Typical target ranges for hemoglobin are, for example, between 11-13 g/dL, or between 10.5-12 g/dL, or between 10.5-13 g/dL. Other acceptable target hemoglobin ranges can be readily determined by competent medical practitioners.

In additional embodiments, effective treatment regimes for compounds of the invention include administration one, two or three times weekly; preferably two or three times weekly. The dosing interval may be altered during the course of treatment, for example, the compound may be administered three times weekly initially for a number of weeks and then administered two times weekly.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### Treatment of Chronic Kidney Disease Patients with Compound A Increases Mean Hemoglobin Levels without Increasing Platelet Counts

Human subjects with stage 3 or 4 chronic kidney disease and stable hemoglobin levels at or below 10.5 g/dL at screening were treated with orally administered compound A as outlined below for 16 weeks (groups A and B) or 24 weeks (groups C and D). No intravenous iron administration was allowed during the treatment, and subjects who had received more than one administration of IV iron within 12 weeks prior to randomization were excluded. Subjects were not on dialysis and had not received ESA therapy within 12 weeks prior to treatment with compound A.

Groups A (n=24) and B (n=24) received an initial weight-adjusted dose of 60 mg (for subjects of 40 to 60 kg), 100 mg (for subjects of >60 to 90 kg), or 140 mg (for subjects of >90 to 140 kg), three times a week for 4 weeks. Treatment was continued for weeks 5 through 16 with dose adjusted every 4 weeks to maintain hemoglobin of 11-13 g/dL (target Hb range). Group A continued dosing three time a week, Group B was switched to twice weekly dosing.

Groups C (n=24) received a initial fixed dose of 50 mg, three times a week for 4 weeks and group D (n=24) received an initial fixed dose of 100 mg, three times a week. Treatment was continued for weeks 5 to 24 with dose adjusted to maintain hemoglobin of 10.5-12 g/dL (target Hb range). Hemoglobin and platelet counts were measured every 4 weeks.

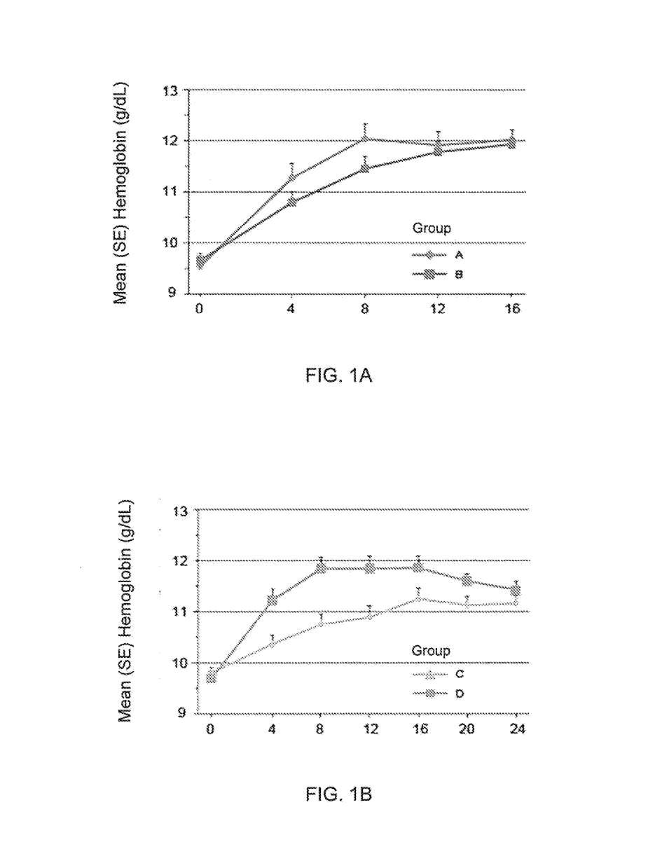

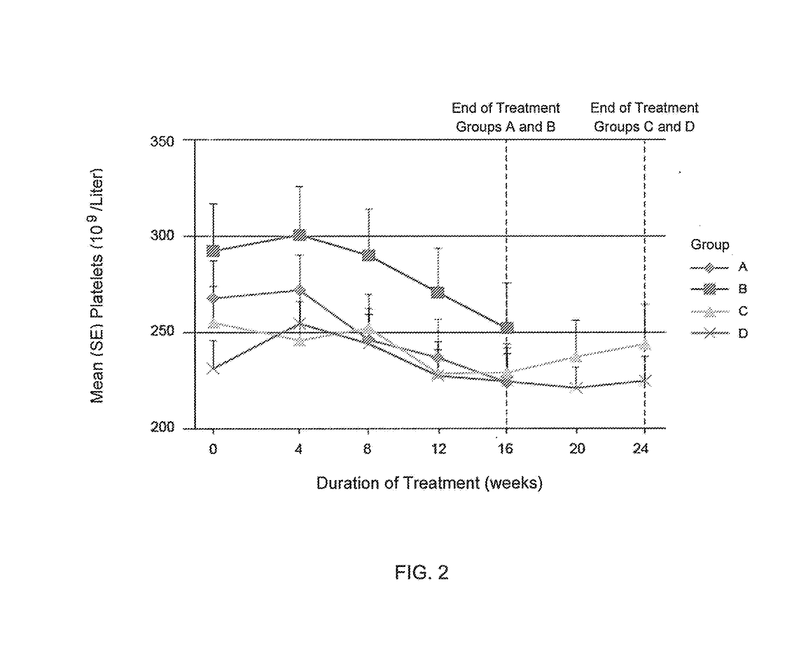

Mean hemoglobin concentrations increased from the baseline in all groups (all p values were <0.0001 end of treatment vs. baseline) in a dose-dependent manner and were maintained within target Hb ranges from week 6 until the end of treatment (FIGS. 1A and 1B). Mean platelet counts decreased significantly from baseline to the end of treatment in Groups A and B (−46.55 and −43.67 1×10⁹/L, respectively; p<0.0001 for both vs. baseline) or decreased slightly from baseline to end of treatment in Groups C and D (−1.69 and −11.88 1×10⁹/L respectively; p=0.89 and p=0.30 vs. baseline) (FIG. 2). Mean baseline platelet counts were 267 1×10⁹/L 292 1×10⁹/L, 255 1×10⁹/L, and 231 1×10⁹/L, for Groups A, B, C, and D, respectively. For the combined groups, the mean change in platelet count from baseline to end of treatment was −30.05 at week 17, p<0.0001; and −6.94 at week 25, p=0.41. Although platelet count decreased from baseline in all groups, platelet count remained within the normal range.

### Example 2

### Treatment of Chronic Kidney Disease Patients with Compound a Decreases the Platelet Counts in Patients Having a Baseline Platelet Count at the High Range of Normal

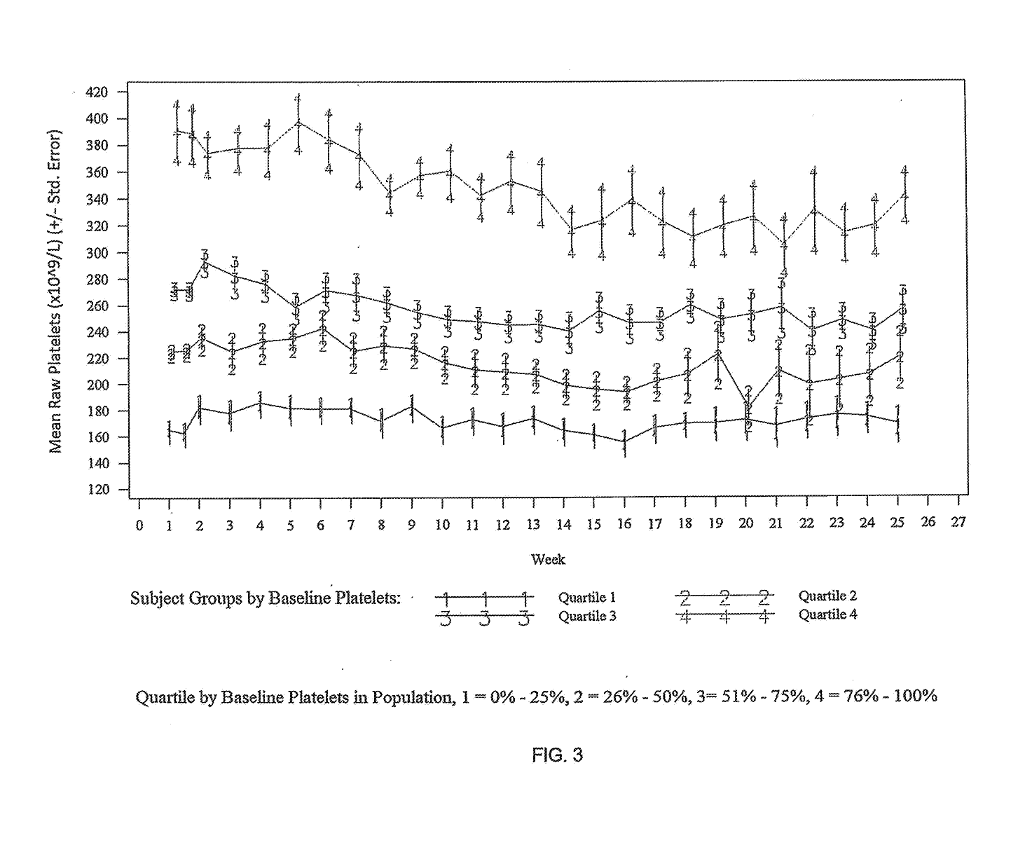

In a separate analysis of the data from Example 1, all patient data on platelet count over time was combined and stratified into quartiles based on the baseline platelet count. Each quartile had n=24. Quartile 1 included the 24 patients with the lowest baseline platelet count. Quartile 2 included the 24 patients with the next highest baseline platelet count after the patients in quartile 1. Quartile 3 included the 24 patients with the next highest baseline platelet count after the patients in quartile 2. Quartile 4 included the 24 patients with the highest baseline platelet count. The patients in quartile 1 had a mean baseline platelet count of 164.9×10⁹/L. The patients in quartile 2 had a mean baseline platelet count of 224.8×10⁹/L. The patients in quartile 3 had a mean baseline platelet count of 272.2×10⁹/L. The patients in quartile 4 had a mean baseline platelet count of 388.7×10⁹/L. The results can be seen in FIG. 3. For patients in the lower 3 quartiles, the platelet count was maintained at (or not significantly increased from) the baseline level throughout the course of treatment. For the patients in the highest quartile of baseline platelet count, quartile 4, the platelet count decreased from baseline during the course of treatment. Compound A effects a greater reduction on the platelet count for patients having a higher baseline platelet count, and does not significantly effect the platelet count for patients having a baseline platelet count in the low range of normal. This demonstrates that Compound A is effective for decreasing platelet count and increasing hemoglobin level for patients having a baseline platelet count in the high range of normal.

These results demonstrate that the methods and compounds of the invention can provide effective treatment for anemia and/or increase hemoglobin without significantly increasing platelet count. The methods of the invention provide effective treatment for anemia while maintaining or decreasing the platelet count of the treated subjects. The methods of the invention provide for an increase in hemoglobin while maintaining or decreasing the platelet count of the treated subjects.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

1. (canceled)

2. (canceled)

3. A method of maintaining the platelet count in a subject in need of treatment for anemia, the method comprising administering to said subject a therapeutically effective amount of a compound that inhibits HIF prolyl hydroxylase, wherein the compound is (4-hydroxy-1-methyl-7-phenoxy-isoquinoline-3-carbonyl)-amino]-acetic acid.

4. (canceled)

5. (canceled)

6. (canceled)

7. (canceled)

8. (canceled)

9. The method of claim 3, wherein the subject is a human subject.

10. The method of claim 9, wherein the human subject has a disorder or condition selected from the group consisting of chronic kidney disease, end stage renal disorder, anemia, low hemoglobin, diabetes, cancer, infection, inflammation, ulcers, an immunosuppressive condition, and polycystic kidney disease.

11. The method of claim 9, wherein the human subject has a baseline pre-treatment hemoglobin level of 11.0 g/dL or less, of 10.5 g/dL or less, of 10.0 g/dL or less, of 9.5 g/dL or less, of 9.0 g/dL or less, or of 8.5 g/dL or less.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. The method according to claim 3, wherein the subject has a baseline platelet count between 150,000/μl and 300,000/μl.

24. The method according to claim 3, wherein the subject has a baseline platelet count of between 300,000/μl and 400,000/μl.

25. (canceled)
